(19) United States
(12) Patent Application Publication
(10) Pub. No.: US 2004/0052800 A1
(43) Pub. Date: Mar. 18, 2004

(54) Stabilization of immunogens derived from paramyxoviruses

(76) Inventors: Bruce Carpick, Mississauga (CA); Bernard Cornet, Toronto (CA); Mathilde Luciani, Richmond Hill (CA)

Correspondence Address: DR. GAVIN ZEALEY, AVENTIS PASTEUR LTD., 1755 STEELES AVE. WEST, TORONTO, ON, M2R 3T4, CA

(21) Appl. No.: 10/343,821

(22) PCT Filed: Aug. 2, 2001
(86) PCT No.: PCT/CA01/01108

(51) Int. Cl. A61K 39/00; A61K 39/38

(52) U.S. Cl. 424/184.1

(57) ABSTRACT

Subunit non-adjuvanted immunogenic compositions of respiratory syncytial virus were stabilized by the formulation with various stabilizing agents including monosodium glutamate. The formulations retained protein conformation for at least 8 weeks at elevated temperatures.

### FIELD OF INVENTION

This invention relates to the field of immunology and is particularly concerned with the stabilization of antigens derived from paramyxoviruses.

### BACKGROUND TO THE INVENTION

The family Paramyxoviridae describe an extremely broad array of viruses which cause numerous serious infections. These infections include mumps, Newcastle disease, measles, canine distemper, rinderpest, various parainfluenza viruses like Sendai virus and simian virus, and Respiratory Syncytial Virus. Human respiratory syncytial virus, (RSV) for example, is the main cause of lower respiratory tract infections among infants and young children (refs. 1-3—a list of references appears at the end of the disclosure and each of the references in the list is incorporated herein by reference thereto).

Paramyxoviruses, like all negative-strand RNA viruses, comprise two structural modules: an internal ribonucleoprotein core called “the nucleocapsid” and an outer, roughly spherical lipoprotein envelope. The nucleocapsid contains a single-stranded viral RNA genome. Paramyxoviruses are generally 150 to 250 nm in diameter, but even larger virus particles are quite common. Some paramyxoviruses are shaped like filaments and are larger as a result. These variations, or pleomorphisms, reflect a relative lack of stringency in the budding stage of the virus assembly process yielding virus particles that possess two or more genome equivalents.

The surfaces of paramyxoviruses have a fuzzy appearance by negative staining because of stalk-like glycoprotein complexes that mediate virus attachment and penetration. Virus envelopes are tough enough to provide effective protection in the transport of nucleocapsids from cell to cell, but they often do not withstand the stresses of drying on electron microscope grids. Hence, they often rupture spontaneously, either releasing the nucleocapsids or permitting the negative stain to enter and outline the morphology of the nucleocapsid.

The helical symmetry of paramyxovirus nucleocapsids is made especially obvious by the large size of the protein structure units, giving the edges of the rods a serrated appearance. The “spiral-staircase” nature of the nucleocapsid helix produces an empty central core in the rod, which can be penetrated by a negative stain.

Environmental conditions, such as ionic composition and pH, affect the flexibility of paramyxovirus nucleocapsids. Such flexibility undoubtedly enables a nucleocapsid to meet two biological requirements: (1) coiling into a form that is compact enough to fit into a unit-size virion envelope and (2) exposing the RNA within to the attention of enzymes that use the RNA as a template. When environmental conditions cause the nucleocapsid to lose flexibility, its full length relative to the diameter of a unit virion is easily appreciated. Each genome of a paramyxovirus occupies a single nucleocapsid, and each paramyxovirus genome contains a complete set of six or more viral genes covalently linked in tandem. The protein structure units of paramyxovirus nucleocapsids protect the associated RNA completely from added ribonuclease.

Those skilled in the art also will recognise Respiratory Syncytial Virus as a representative example of the morphology of paramyxoviruses. The structure and composition of Respiratory Syncytial Virus have been elucidated and described in detail in the textbook “Fields Virology”, Fields, B. N. et al. Raven Press, N.Y. (1996), in particular, Chapter 54, pp 1285-1304 “Respiratory Syncytial Virus” by P., McIntosh, K., and Chanock, R. M. (ref. 4).

Respiratory Syncytial Virus is one of the most important causes of lower respiratory tract illness in infants two to six months of age and children (ref. 5). In the USA alone, 100,000 children may require hospitalization for pneumonia and bronchitis caused by Respiratory Syncytial Virus in a single year (refs. 6, 7); and providing inpatient and ambulatory care for children with Respiratory Syncytial Virus infections costs in excess of $340 million annually (ref 8). More importantly, approximately 4,000 infants in the USA die each year from complications arising from severe respiratory tract disease caused by Respiratory Syncytial Virus and Parainfluenza type 3 virus infection. Further, 65 million infections occur globally every year resulting in 160,000 deaths (ref 9). The World Health Organization and the National Institute of Allergy and Infectious Disease vaccine advisory committees have ranked Respiratory Syncytial Virus second only to HIV for vaccine development.

Respiratory Syncytial Virus infection in adults was initially considered a significant problem only in certain high-risk populations, such as the institutionalized elderly. However, evidence has been accumulating that the infection occurs frequently in previously healthy adults (ref. 10). Respiratory Syncytial Virus infections in the elderly usually represent reinfections in those who have had many prior episodes. These infections have been reported to cause altered airway resistance and exacerbation of chronic obstructive lung disease. In adults over 60 years old, Respiratory Syncytial Virus usually causes mild nasal congestion and may also result in fever, anorexia, pneumonia, bronchitis and death (ref. 11).

A vaccine is not yet available even though the importance of RSV as a respiratory pathogen has been recognized for over 30 years. Several strategies have been used in RSV vaccine development including inactivation of the virus with formalin (ref. 19), isolation of cold adapted and/or temperature-sensitive mutant viruses (ref 20) and purified F or G glycoproteins (refs. 21, 22, 23). Clinical trial results have shown that both live attenuated and formalin-inactivated vaccines failed to adequately protect against RS virus infection (ref. 24). Thus a subunit vaccine producing the proper immune response is desirable.

The two major protective antigens from RSV that induce virus neutralizing antibodies are the envelope fusion (F) glycoprotein and the attachment (G) glycoprotein (ref. 12). The F protein is synthesized as a precursor molecule (Fa) about 68 kDa which is proteolytically cleaved into two disulfide-linked polypeptide fragments, F1 about 48 kDa and F2 about 20 kDa (ref. 13). The G protein about 33 kDa is heavily O-glycosylated giving rise to a glycoprotein having a molecular weight of about 90 kDa (ref. 14). Two broad subtypes of Respiratory Syncytial Virus have been defined as A and B (ref. 15). The major antigenic differences between these subtypes are found in the G glycoprotein while the F glycoprotein is more conserved (refs. 8, 16). In addition to the antibody response generated by the F and G glycoproteins, human cytotoxic T cells produced by Respiratory Syncytial Virus infection have been shown to recognize matrix protein (M), nucleoprotein (N), and small hydrophobic protein SH (ref. 17).

U.S. Pat. No. 6,020,182, the disclosures of which are incorporated herein by reference, issued Feb. 1, 2000, teaches the combination of a mixture of Respiratory Syncytial Virus proteins in a vaccine formulation to provide an immune response which is substantially the same as that obtained by administration of the components individually. Accordingly, there is no observed detrimental effect on the immunogenicity of the individual components by combining them in a single formulation. Consequently, immunogenic compositions for conferring protection in a host against disease caused by Respiratory Syncytial Virus may comprise an immunoeffective amount of a mixture of purified fusion (F) protein, attachment (G) protein and matrix (M) protein of Respiratory Syncytial Virus. The immunogenic composition preferably is formulated as a vaccine for In vivo administration to the host.

The Respiratory Syncytial Virus protein mixture employed by those skilled in the art, when analyzed by reduced SDS-PAGE analysis, may comprise fusion (F) protein with an F1 subunit of approximately 48 kDa and an F2 subunit of approximately 23 kDa; attachment (G) protein with a G1 subunit of approximately 95 kDa and a G2 subunit of approximately 55 kDa; and matrix (M) protein with an M subunit of approximately 31 kDa. When analyzed by SDS-PAGE under reducing conditions and densitometric scanning following silver staining, the ratio of F1 subunit of molecular weight approximately 48 kDa to F2 subunit of molecular weight approximately 23 kDa in this mixture may be approximately between 1:1 and 2:1. The Respiratory Syncytial Virus proteins provided in the mixture generally are substantially non-denatured by the mild conditions of preparation and may comprise Respiratory Syncytial Virus proteins from one or both of subtypes Respiratory Syncytial Virus A and Respiratory Syncytial Virus B. ibid.

Stabilizing these kinds of protein compositions is difficult but necessary if they are to be useful. Generally, protein instability can be divided into two forms, chemical instability and physical instability. Chemical instability relates to processes that involve the formation or destruction of covalent bonds which lead to new chemical entities. These processes include reactions such as proteolysis, deamidation, racemization, oxidation, and elimination. Physical instability refers to any change of state that does not include bond cleavage or formation and is most relevant to the stabilization of immunogens derived from paramyxoviruses.

Physical instability relates to a number of processes the most common of which is denaturation, or loss of the higher-order, globular structure that proteins adopt upon folding. Physical instability also includes aggregation, precipitation, and adsorption to surfaces. Aggregation is defined as a microscopic process whereby protein molecules associate. These aggregates may be as small as dimers (as with insulin) or large primary particles, which occur as intermediates in the precipitation process. In either case, the aggregates remain in solution and are not visible to the naked eye. The activity and immunogenicity of the aggregates may be different from the native protein. Precipitation, on the other hand, is a macroscopic process, producing a visible material. This may take the form of an amorphous solid, fibrils, crystals, or simply clouding of the solution. Adsorption is the association of proteins with surfaces rather than each other, although both may occur. The surface may be a solid, such as a container or column, or it may be a gaseous interface, such as at the air-water interface. All of the above types of physical processes have dramatic consequences for pharmaceutical scientists attempting to handle biotechnology products. (ref. 18).

Pharmaceutical scientists are faced with greater and more complex formulation challenges as the tremendous expansion in molecular biology and immunology produces an ever larger number of new and novel immunogens and as advances in biochemistry continue to increase the purity of immunogens. Furthermore, in addition to formulations for the more traditional injectable products, there is now a demand for alternate dosage forms. What makes this particularly challenging is that increased purification of these products removes them ever further from their most stable natural environments.

Solvent protein interaction strongly influences conformation. Polypeptide chains of more than 50 linear amino acids have secondary, tertiary, and sometimes quaternary structural conformations. The structure of these chains greatly effects their overall function, including immunogenicity. Further, each of the 20 amino acids has its own functional group side chain, and both secondary and tertiary structure result from the sequence and interaction of these side chains. Water, the normal solvent of proteins, forms a hydration layer around the protein and contributes to hydrogen bonding. The hydrophobic interaction of the amino acid hydrocarbon side chains buried within the molecule provides additional stabilization energy. Generally, because proteins exist in an aqueous environment, the folding of the molecule results in the internal burial of hydrophobic groups and the surface exposure of hydrophilic groups. This results in an energetically favorable state.

The purification process strips away contaminates, such as carbohydrates; salts; lipids; and other proteins, that keep immunogenic protein subunits neatly folded into thermodynamically favorable shapes. When such contaminates are removed, new hydrophobic and hydrophilic areas are exposed changing the overall thermodynamics of the immunogen and causing it to change shape. Disruption of the structure occurs when the solvent partitions hydrocarbon side chains between the hydrocarbon phase and the aqueous phase. A highly purified protein subunit is rendered more sensitive to processes such as shear, agitation, enzymatic and chemical degradation, and aggregation because the removal of contaminates changes the thermodynamics of the subunit.

It is the tertiary structure of protein subunit immunogens in particular that must be stabilized against various disruptive forces that occur during processing and handling. Potential denaturing forces include both chemical and physical stress. Chemical stress results from factors used in purification such as pH, ionic strength, and detergents. Physical stress results from filtration and filling where surface adsorption and shear contribute to the unwinding of tertiary structure into a random coil.

As described above, paramyxoviruses are prevalent and cause terrible infections. It would be desirable to confer protection against such infections with the administration of a vaccine. Such a vaccine might be composed from at least one effective immunogen derived from a paramyxovirus. It would facilitate the use of these immunogens if they could be stabilized and stored at temperatures above freezing so as not to lose their conformational structures. However, many issues and concerns combine to make the prediction and discovery of suitable formulation excipients and processes very difficult, and a method to stabilize immunogens derived from paramyxoviruses is unknown.

### SUMMARY OF THE INVENTION

The inventors have surprisingly discovered that immunogens derived from paramyxoviruses are stabilized when formulated with monosodium glutamate (MSG). Monosodium glutamate is known to those skilled in the art as a monosodium salt of the naturally occurring L-form of glutamic acid is manufactured by fermentation of carbohydrate sources such as sugar beet molasses. The Chinese have used monosodium glutamate as a seasoning in foods for centuries. This white, practically odorless material takes the form of crystals or a free-flowing crystalline powder and is very soluble in water. The present invention is directed toward the stabilization of paramyxovirus antigenic preparations, such as Respiratory Syncytial Virus vaccine, with monosodium glutamate.

In accordance with one aspect of the invention, there is provided an antigenic preparation comprised of at least one immunogen derived from a paramyxovirus and a predetermined concentration of monosodium glutamate as a stabilizer therefor. The immunogen may be derived from the paramyxovirus Respiratory Syncytial Virus. Predetermined concentrations of monosodium glutamate include about between 0.12 and 20% wt/v.

In one embodiment of the present invention, where the at least one immunogen is derived from Respiratory Syncytial Virus, the at least one immunogen may be selected from the group consisting of fusion (F) protein, attachment (G) protein, and matrix (M) protein. Further, the subunit proteins in the aforementioned group may be purified. In another aspect of this invention, the at least one immunogen may be derived from Respiratory Syncytial Virus type A or B, and the newly stabilized antigenic preparations may include sucrose.

The present invention also includes a vaccine formulated from an antigenic preparation comprising at least one immunogen derived from a paramyxovirus and a predetermined concentration of monosodium glutamate as a stabilizer therefor. In addition, the invention includes a method for producing a stable vaccine comprising the steps of combining at least one immunogen derived from a paramyxovirus and a predetermined concentration of monosodium glutamate as a stabilizer therefor. The immunogen may be derived from the paramyxovirus, Respiratory Syncytial Virus. Predetermined concentrations of monosodium glutamate include about between 0.12 and 20% (wtv).

In a further aspect of the invention, when the at least one immunogen is derived from Respiratory Syncytial Virus, the at least one immunogen may be selected from the group consisting of fusion (F) protein, attachment (G) protein, and matrix (M) protein. Further, the subunit proteins in the aforementioned group may be purified. In addition, the at least one immunogen may be derived from Respiratory Syncytial Virus type A or B, and the newly stabilized vaccines may be combined with a suitable adjuvant.

The invention also extends to a method of generating an immune response in a host, including human, comprising administering thereto an immuno-effective amount of the immunogenic compositions provided here in.

### BRIEF DESCRIPTION OF THE DRAWINGS

FIG. 1 shows a graph demonstrating RSV A M ELISA activity over time at 5° C., 25° C. and 37° C. in the unformulated RSV preparation described in example 1.

FIG. 2 shows a graph demonstrating the stabilizing effect of 1M monosodium glutamate on the M ELISA activity of the RSV preparation described in example 1 for at least eight weeks at 25° C.

FIG. 3 shows a graph demonstrating the stabilizing effect of monosodium glutamate compared to other stabilizers at maintaining RSV M ELISA activity for at least eight weeks at 25° C.

FIG. 4 shows a graph demonstrating the stabilizing effect of 1 M sodium glutamate on RSV F protein ELISA activity at 25 and 37° C.

FIG. 5 shows a graph demonstrating the M ELISA stability effect of 5% sucrose and 10% MSG at 25° C. and 37° C.

### GENERAL DESCRIPTION OF INVENTION

Immunogens derived from paramyxoviruses are stabilized when combined with monosodium glutamate. The immunogens may be isolated and purified from paramyxoviruses like Respiratory Syncytial Virus.

Studies were initiated on RSV A non-adjuvanted vaccine because of an observation of a rapid loss of M protein antigen ELISA reactivity in stability studies at elevated temperature, and a gradual loss over time (6 months to 1 year) at 2-8° C. This loss of reactivity was not directly correlated with a loss of M protein (as judged by SDS-PAGE and Western blot), and thus likely involved a change in protein conformation. The experiments show that the addition of monosodium glutamate increases the stabilization of the M ELISA, F ELISA, and the F and M proteins as judged by SDS-PAGE.

1. Vaccine Preparation and Use

Immunogenic compositions, suitable to be used as vaccines, may be prepared from mixtures comprising immunogenic F, G and M proteins of RSV. The immunogenic composition elicits an immune response which produces antibodies, including anti-RSV antibodies which are anti-F, anti-G and anti-M antibodies.

Immunogenic compositions including vaccines may be prepared as injectables, as liquid solutions, suspensions or emulsions. The active immunogenic ingredients may be mixed with pharmaceutically acceptable excipients, which are compatible therewith.

Such excipients may include water, saline, dextrose, glycerol, ethanol and combinations thereof. The immunogenic compositions and vaccines may further contain auxiliary substances, such as, wetting or emulsifying agents, pH buffering agents, or adjuvants to enhance the effectiveness thereof. Immunogenic compositions and vaccines may be administered parentally, by injection subcutaneous, intradermal or intramuscularly injection. Alternatively, the immunogenic compositions formulated according to the present invention, may be formulated and delivered in a manner to evoke an immune response at mucosal surfaces. Thus, the immunogenic composition may be administered to mucosal surfaces by, for example, the nasal or oral (intagastric) routes. Alternatively, other modes of administration including suppositories and oral formulations may be desirable. For suppositories, binders and carriers may include, for example, polyalkalene glycols or triglycerides. Such suppositories may be formed from mixtures containing the active immunogenic ingredient(s) in the range of about 10%, preferably about 1 to 2%. Oral formulations may include normally employed carriers, such as, pharmaceutical grades of saccharine, cellulose and magnesium carbonate. These compositions can take the form of solutions, suspensions, tablets, pills, capsules, sustained release formulations or powders and contain about 1 to 95% of the active ingredients, preferably about 20 to 75%.

The immunogenic preparations and vaccines are administered in a manner compatible with the dosage formulation, and in such amount as will be therapeutically effective, immunogenic and protective. The quantity to be administered depends on the subject to be treated, including, for example, the capacity of the individual's immune system to synthesize antibodies, and, if needed, to produce a cell-mediated immune response. Precise amount of active ingredients required to be administered depend on the judgment of the practitioner. However, suitable dosage ranges are readily determinable by one skilled in the art and may be of the order of micrograms to milligrams of the active ingredient(s) per vaccination. Suitable regimes for initial administration and booster doses are also variable, but may include an initial administration followed by subsequent booster administrations. The dosage may also depend on the route of administration and will vary according to the size of the host.

The concentration of the active ingredients in an immunogenic composition according to the invention is in general about 1 to 95%. A vaccine which contains antigenic material of only one pathogen is a monovalent vaccine.

Immunogenicity can be significantly improved if the antigens are co-administered with adjuvants. Adjuvants enhance the immunogenicity of an antigen but are not necessarily immunogenic themselves. Adjuvants may act by retaining the antigen locally near the site of administration to produce a depot effect facilitating a slow, sustained release of antigen to cells of the immune system. Adjuvants can also attract cells of the immune system to an antigen depot and stimulate such cells to elicit immune responses.

Immunostimulatory agents or adjuvants have been used for may years to improve the host immune responses to, for example, vaccines. Intrinsic adjuvants, such as, lipopolysaccharides, normally are the components of the skilled or attenuated bacteria used as vaccines. Intrinsic adjuvants are immunomodulators which are formulated to enhance the host immune responses. Thus, adjuvants have been identified that enhance the immune responses to antigens delivered parentally. Some of these adjuvants are toxic, however, and can cause side effects, making them unsuitable for use in humans and many animals. Indeed, only aluminum hydroxide and aluminum phosphate (collectively commonly referred to as alum) are routinely used as adjuvants in human and veterinary vaccines. The efficacy of alum in increasing antibody responses to diphtheria and tetanus toxoids is well established. While the usefulness of alum is well established for some applications, it has limitations. For example, alum is ineffective for influenza vaccination and usually does not elicit a cell mediated immune response. The antibodies elicited by alum-adjuvanted antigens are mainly of the IgG1 isotype in the mouse, which may not be optimal for protection by some vaccinal agents.

A wide range of extrinsic adjuvants can provoke potent immune responses to antigens. These include saponins complexed to membrane protein antigens (immune stimulating complexes), pluronic polymers with mineral oil, killed mycobacteria in mineral oil, Freund's incomplete adjuvant, bacterial products, such as, muramyl dipeptide (MOP) and lipopolysaccharide (LPS), as well as lipid A, and liposomes.

To efficiently induce humoral responses (HIR) and cell-mediated immunity (CMI), immunogens are often emulsified in adjuvants. Many adjuvants are toxic, including granulomas, acute and chronic inflammations (Freund's complete adjuvant, FCA), cytolysis (saponins and Pluonic polymers) and pyrogenicity, arthritis and anterior uveitis (LPS and MOP). Although FCA is an excellent adjuvant and widely used in research, it is not licensed for use in human or veterinary vaccines because of its toxicity.

### EXAMPLES

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient Although specific terms have been employed herein, such terms are intended in a descriptive sense and not for purposes of limitation.

### Example 1

This Example illustrates how the immunogens used herein may be isolated and purified from paramyxoviruses like Respiratory Syncytial Virus. In the case of Respiratory Syncytial Virus, as described in U.S. patent Ser. No. 08/679,060 and WO 98/02457, the virus is grown on a vaccine quality cell line such as VERO cells and on human diploid cells such as MRC5 and WI38 and then harvested. Fetal bovine serum (FBS) and trypsin may effect fermentation.

The viral harvest is filtered and then concentrated, typically using tangential flow ultrafiltration with a membrane of desired molecular weight cut-off, and diafiltered. The virus harvest concentrate may be centrifuged and the supernatant discarded. The pellet following centrifugation may be washed with a buffer containing urea to remove soluble contaminants while leaving the F, G and M proteins substantially unaffected, and the resulting material then may be recentrifuged. The pellet from the centrifugation then is detergent extracted to solubilize the F, G and M proteins from the pellet Such detergent extraction may be effected by resuspending the pellet to the original harvest concentrate volume in an extraction buffer. Such a buffer would be a detergent, like TRITON™ X-100, a non-ionic detergent which is octadienyl phenol (ethylene glycol) 10. Other detergents include octylglucoside and Mega detergents.

Following centrifugation to remove non-soluble proteins, the F, G and M protein extract is purified by chromatographic procedures. The extract may first be applied to an ion exchange chromatography matrix to permit binding of the F, G and M-proteins to the matrix while impurities are permitted to flow through the column. Although the ion-exchange chromatography matrix may be any desired chromatography material, materials with a calcium phosphate matrix, specifically hydroxyapatite, were found to work well. DEAE and TMAE were commonly used in these protocols. The bound F, G and M proteins then are coeluted from the column by a suitable eluant. The resulting copurified F, G and M proteins may be further processed to increase the purity thereof. The purified F, G and M proteins employed herein may be in the form of homo and hetero oligomers including F:G heterodimers and including dimers, tetramers and higher species. The Respiratory Syncytial Virus protein preparations prepared following this procedure demonstrated no evidence of any adventitious agent, hemadsorbing agent or live virus.

### Example 2

This Example illustrates the formulation of immunogens.

Initial screening experiments were performed on non-adjuvanted Respiratory Syncytial Virus B, with promising formulations applied to subsequent studies using Respiratory Syncytial Virus A, also non-adjuvanted. Similar activity profiles were observed for all formulations tested on both Respiratory Syncytial Virus A and Respiratory Syncytial Virus B. Samples were diluted to approximately 200 μg/ml Respiratory Syncytial Virus A protein mixture, divided into formulation lots and then combined with an equal volume of formulation stock solution containing the excipient at twice the final concentration.

### Example 3

This Example illustrates measurement of stability for the various formulations.

Each formulation lot was divided into aliquots of approximately 0.5 ml, and these samples were incubated at various temperatures (−70° C., 5° C., 25° C., 37° C., 45° C.). Samples were tested by SDS-PAGE, Western blot, and ELISA assay for the G, F, and M protein antigens at various time points, depending on the incubation temperature and duration of the study. Samples incubated at −70° C. were used as non-degraded reference standards in the above-mentioned assays.

SDS-PAGE was performed using pre-cast 12% polyacrylamide gels (Novex). Protein bands were visualized either by direct Coomassie staining of the gels, or by electroblot transfer from the gel to a polyvinyldifluoride membrane (Millipore) and subsequent detection by Western blot. For the Western blot, the membrane was probed with a mixture of anti-F, -G, and -M primary antibodies (lot #5353C75, #131-2G, and 197-F or their equivalents, respectively). Antigen ELISA analyses were performed using equivalent antibodies against F, G, and M proteins.

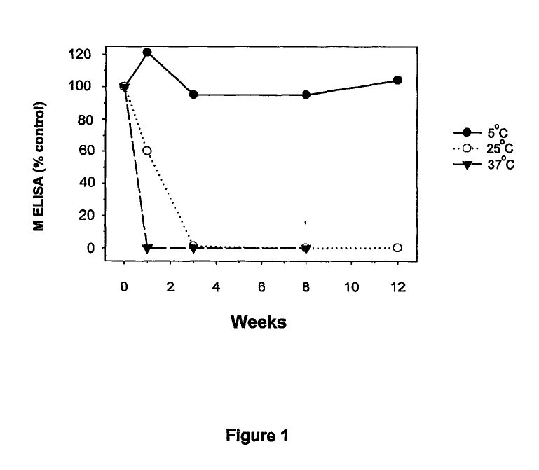

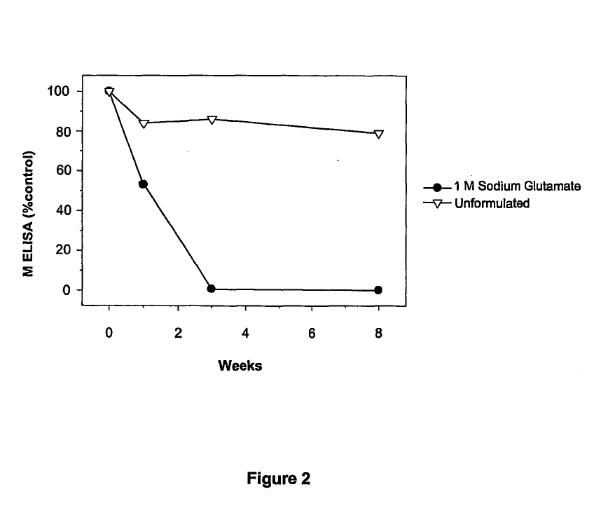

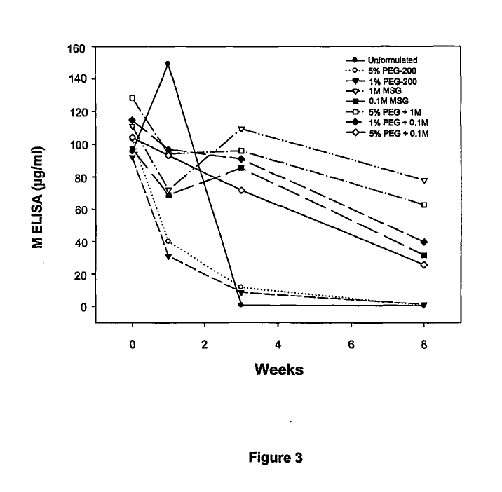

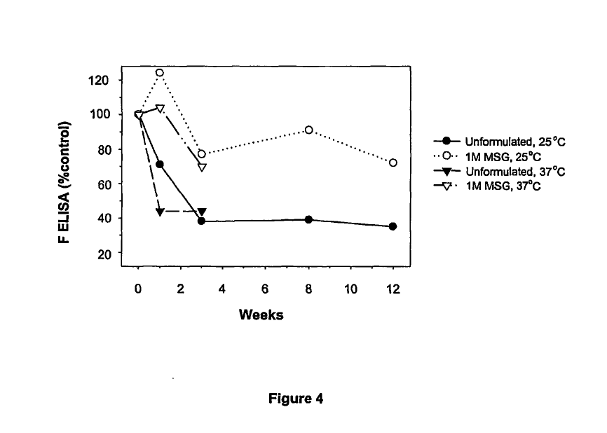

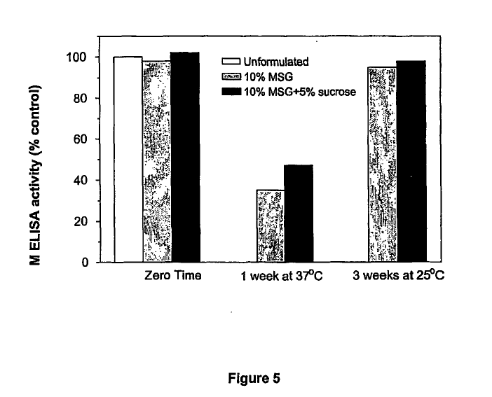

Screening was done using SDS-PAGE, Western blot, and ELISA to identify PEG-200 (1% and 5%, w/v) and sodium glutamate (MSG; 1 M and 0.1 M) as promising stabilizers for the M ELISA. Subsequent experiments showed that 1 M MSG (17%, w/v) showed the best stabilization of the RSV M protein, partially stabilizing M ELISA for 1 week at 37° C., and stabilizing at better than 80% of controls for 8 weeks at 25° C. (FIG. 2). Unformulated samples had zero M ELISA activity under these conditions (FIG. 1). Optimization experiments showed that a lower concentration (0.59 M; 10%, w/v) of MSG achieved similar results (FIG. 3), and that the effect was potentiated by the addition of 5% (w/v) sucrose (FIG. 5). In addition to the observed stabilization of the M protein as assayed by ELISA, the stability of F ELISA (FIG. 4), and of the F and M proteins (as judged by SDS-PAGE) was also improved by these formulations. The percentage of ELISA activity was measured in relation to samples stored at −70 C as control standards.

### SUMMARY OF THE DISCLOSURE

In summary of the disclosure, the present invention provides an antigenic preparation comprising at least one immunogen derived from a paramyxovirus and a predetermined concentration of monosodium glutamate. The preparation may also contain other stabilizing agents.

### REFERENCES

1. Glezen, W. P., Paredes, A. Allison, J. E., Taber, L. H. and Frank, A. L. (1981). J. Pediatr. 98, 708-715.

2. Chanock, R. M., Parrot, R. H., Connors, M., Collins, P. L. and Murphy, B. R. (1992). Pediatrics 90, 137-142.

3. Martin, A. J. Gardiner, P. S. and McQuillin, J. (1978). Lancel ii, 1035-1038.

4. Collins, P., McIntosh, K., and Chanock, R. M. in “Fields Virology” ed. by Fields, B. N., Knipe, D. M., and Howley, P. M., Lippincott-Raven Press, New York, (1996) pp. 1313-1351.

5. McIntosh, K. and Chanock, R. M. (1990) in Fields Virology (Fields, B. M., and Knipe, D. M. eds.) pp. 1045-1075, Raven Press, Ltd., New York.

6. Glezen, W. P., Taber, L. H., Frank, A. L. and Kasel, J. A. (1986) Am. J. Dis. Child. 140, 143-146.

7. Katz, S. L. New vaccine development establishing priorities. Vol. 1. Washington: National Academic Press. (1985) pp. 397409.

8. Wertz, G. W., Sullender, W. M. (1992) Biotech 20, 151-176.

9. Robbins, A., and Freeman, P. (1988) Sci. Am. 259, 126-133.

10. Murry A R, Dowell S F, 1997. Respiratory syncytial virus: not just for kids. Hosp. Pract. (Off Ed) Jul. 15, 1997; 32(7): 87-8, 914.

11. Mlinaric-Galinovic G, Falsey A R, Walsh E E, 1996. Respiratory syncytial virus infection in the elderly. Eur J Clin Microbiol Infect Dis 1996.

12. Walsh, E. E., Hall, C. B., Briselli, M., Brandiss, M. W. and Schlesinger, J. J. (1987) J. Infect. Dis. 155, 1198-1204.

13. Walsh, E. E., Hruska, J. (1983) J. Virol. 47, 171-177.

14. Levine, S., Kleiber-France, R., and Paradiso, P. R. (1987) J. Gen. Virol. 69, 2521-2524.

15. Anderson, L. J. Hierholzer, J. C., Tsou, C., Hendry, R. M., Fernie, B. F., Stone, Y. and McIntosh, K. (1985), J. infect. Dis. 151, 626-633.

16. Johnson, P. R., Olmsted, R. A., Prince, G. A., Murphy, B. R., Alling, D. W., Walsh, E. E. and Collins, P. L. (1987) J. Virol. 61 (10), 3163-3166.

17. Cherrie, A. H., Anderson, K., Wertz, G. W., and Openshaw, P. J. M. (1992) J. Virology 66, 2102-2110.

18. Ahern, Tim J. and Manning, Mark C. (1992) Stability of Protein Pharmaceuticals, x.

19. Kim, H. W., Canchola, J. G., Brandt, C. D., Pyles, G., Chanock R. M. Jensen, K., and Parrott, R. H. (1969) Amer. J. Epidemiology 89, 422-434.

20. Firedewald, W. T., Forsyth, B. R., Smith, C. B., Gharpure, M. S., and Chanock, R. M. (1968) JAMA 204, 690-694.

21. Walsh, E. E., Brandriss, M. W., Schlesinger, J. J. (1985) J. Gen. Virol. 66, 409-415.

22. Walsh, E. E., Schlesinger, J. J. and Brandriss, M. W. (1984) J. Gen. Virol. 65, 761-766.

23. Routledge, E. G., Willcocks, M. M., Samson, A. C. R., Morgan, L., Scott, R., Anderson, J. J., and Toms, G. L. (1988) J. Gen. Virology 69, 293-303.

24. Fulginiti, V. A., Eller, J. J., Sieber, O. F., Joyner, I. W., Minamitani, M. and Meiklejohn, G. (1969) Am J. Epidemiol. 89 (4), 435-448.

What we claim is:

1. An antigenic preparation comprising at least one immunogen derived from a paramyxovirus and a predetermined concentration of monosodium glutamate as a stabilizer therefor.

2. The antigenic preparation of claim 1 wherein the paramyxovirus is Respiratory Syncytial Virus.

3. The antigenic preparation of claim 1 wherein the concentration of monosodium glutamate is about between 0.12 and 20% wt/v.

4. The antigenic preparation of claim 2 wherein the at least one Respiratory Syncytial Virus immunogen is selected from the group consisting of fusion (F) protein, attachment (G) protein, and matrix (M) protein.

5. The antigenic preparation of claim 1 wherein the at least one immunogen is purified.

6. The antigenic preparation of claim 1 further comprising sucrose.

7. The antigenic preparation of claim 2 wherein the at least one Respiratory Syncytial Virus immunogen is of type A or B.

8. The antigenic preparation of claim 1 formulated as a vaccine for protection against disease caused by paramyxoviruses.

9. A method for preparation of a vaccine comprising the steps of combining at least one immunogen derived from a paramyxovirus and a predetermined concentration of monosodium glutamate as a stabilizer therefor.

10. The method of claim 9 wherein the paramyxovirus is Respiratory Syncytial Virus.

11. The method of claim 9 wherein the concentration of monosodium glutamate is about between 0.12 and 20% wt/v.

12. The method of claim 10 wherein the at least one Respiratory Syncytial Virus immunogen is selected from the group consisting of fusion (F) protein, attachment (G) protein, and matrix (M) protein.

13. The method of claim 12 wherein the at least one Respiratory Syncytial Virus immunogen is purified.

14. The method of claim 9 further comprising sucrose.

15. The method of claim 10 wherein the at least one Respiratory Syncytial Virus immunogen is of type A or B.

16. A method for immunization comprising administering the vaccine of claim 8 to a host.

17. The method of claim 16 wherein the paramyxovirus is Respiratory Syncytial Virus.

18. The method of claim 16 wherein the host is human.

19. The method of claim 17 wherein the at least one Respiratory Syncytial Virus immunogen is type A or B.
